(19) United States
(12) Patent Application Publication
(10) Pub. No.: US 2021/0340509 A1
(43) Pub. Date: Nov. 4, 2021

(54) Reverse transcriptase with increased enzyme activity and application thereof

(71) Applicant: BGI SHENZHEN, Shenzhen (CN)

(72) Inventors: Huanhuan Liu, Shenzhen (CN); Na Guo, Shenzhen (CN); Huizhen Li, Shenzhen (CN); Zhougang Zhang, Shenzhen (CN); Hongyan Han, Shenzhen (CN); Miaomiao Guo, Shenzhen (CN); Yue Zheng, Shenzhen (CN); Yuliang Dong, Shenzhen (CN); Wenwei Zhang, Shenzhen (CN); Chongjun Xu, Shenzhen (CN)

(21) Appl. No.: 17/358,856

(22) Filed: Jun. 25, 2021

Related U.S. Application Data
(63) Continuation of application No. PCT/CN2018/123994, filed on Dec. 26, 2018.

(51) Int. Cl. C12N 9/12; C12N 15/10; C12Q 1/6869; C12N 15/70; C12Q 1/686; C12Q 1/6806

(52) U.S. Cl. CPC C12N 9/1276; C12N 15/1096; C12Q 1/6869; C12Y 207/07049; C12Q 1/686; C12Q 1/6806; C12N 15/70

(57) ABSTRACT

The present disclosure relates to a reverse transcriptase and an application thereof. The reverse transcriptase has mutation sites such as R450H compared with the wild-type M-MLV reverse transcriptase. The reverse transcriptase has increased polymerase activity, improved thermal stability, and reduced RNase H activity.

### PRIORITY INFORMATION

This application is a continuation application of PCT Application No. PCT/CN2018/123994, filed with the China National Intellectual Property Administration on Dec. 26, 2018, the entire content of which is incorporated herein by reference.

### FIELD

The present disclosure relates to the field of enzyme engineering, in particular to a reverse transcriptase with increased enzyme activity and applications thereof, more particularly to a reverse transcriptase with increased polymerization activity, increased thermal stability and decreased RNase H activity.

### BACKGROUND

Reverse transcriptase (RT) is a DNA polymerase that exists in viruses and is responsible for the replication of the viral genome, which has RNA and DNA-dependent DNA polymerase activity and RNase H activity. Use of reverse transcriptase to convert mRNA into cDNA is an important step in the study of gene expression. There are three main types of reverse transcriptases, including reverse transcriptase derived from avian myeloblastosis virus (AMV), reverse transcriptase derived from Moloney murine leukaemia virus (M-MLV), and reverse transcriptase derived from Human Immunodeficiency Virus (HIV). The former two reverse transcriptases are widely used in cDNA synthesis due to their high catalytic activity and relatively high fidelity. Compared to MMLV RT, AMV RT has a reaction temperature which is 3° C.-5° C. higher than that of MMLV RT, but has stronger RNase H activity, which can cause the fragmentation of the RNA template at the 3′-OH end of synthesized cDNA chain, thereby affecting the synthesis of full-length cDNA.

Further research and improvement are required to obtain a reverse transcriptase with high quality requires.

### SUMMARY

The present disclosure aims to solve one of the technical problems in the related art at least to a certain extent. For this, a first aspect of the present disclosure is to provide a reverse transcriptase with improved polymerase activity, improved thermal stability and decreased RNase H activity, so that the provided reverse transcriptase has high polymerase activity, high thermal stability and low RNase H activity.

In the reverse transcription reaction involving reverse transcriptase, increasing the reaction temperature can benefit to unlocking the secondary structure of the RNA template and reducing the non-specific binding of primer to template. However, reverse transcriptase is a normal temperature enzyme, which is easily denatured and inactivated at a high temperature. Therefore, increasing the heat resistance of reverse transcriptase can not only effectively synthesize cDNA, but also facilitate the storage, packaging and transportation of the reverse transcriptase. At the same time, reverse transcriptase has two activities: DNA polymerase activity and RNase H activity, in which RNase H activity would shorten the length of synthesized cDNA and reduce the efficiency of reverse transcription, further the removal of RNase H activity can significantly enhance the thermal stability of reverse transcriptase. Therefore, it is of great significance to obtain a reverse transcriptase with high thermal stability and high polymerase activity for use in reverse transcription reactions by studying the reverse transcriptase derived from M-MLV that lacks RNase H activity.

Thus, in a first aspect of the present disclosure, provided in embodiments is a reverse transcriptase, comprising at least one of mutations from R450H, E286K-E302K-W313F-D524A-D583G, T306K-D583G, E562K-D583N, W313F-D524G-D583N, T306K-D524A, E302K-D524A, E302K-L435R-D524A, L435G-D524A, E302K-L435R-D524A-E562Q, E302K-L435G-D524A, D524G-R450H, W313F-D524A, W313F-E562K-D583N, D583N-E562Q, E286K-E302K-W313F-T330P-D524A-D583G, D524G-D583N-R450H, E302R-W313F-L435G and W313F-L435G, compared to the amino acid sequence of SEQ ID NO: 2.

The reverse transcriptase provided in the present disclosure has improved polymerase activity, improved thermal stability and decreased RNase H activity compared to the wild-type reverse transcriptase, thus can be useful in reverse transcription reactions with low template starting amount and cDNA library construction in single cell sequencing.

In some embodiments of the present disclosure, the above-mentioned reverse transcriptase may further have the following technical features.

In some embodiments of the present disclosure, the reverse transcriptase has increased polymerase activity and decreased RNase H activity.

In some embodiments of the present disclosure, a polymerase activity of the reverse transcriptase is at least 1 to 4 times higher than that of a wild-type M-MLV reverse transcriptase.

In some embodiments of the present disclosure, an RNase H activity of the reverse transcriptase is reduced by 30% to 80% compared to that of a wild-type M-MLV reverse transcriptase.

In some embodiments of the present disclosure, the reverse transcriptase keeps its reverse transcriptase activity unchanged after being heated at 50° C. for 30 minutes.

In some embodiments of the present disclosure, the reverse transcriptase keeps its reverse transcriptase activity unchanged after being heated at 42° C. for 30 minutes.

According to a second aspect of the present disclosure, provided in embodiments is an isolated nucleic acid molecule encoding the reverse transcriptase as described in the first aspect.

According to a third aspect of the present disclosure, provided in embodiments is a construct comprising the isolated nucleic acid molecule as described in the second aspect.

In some embodiments of the present disclosure, the construct is a plasmid.

In some embodiments of the present disclosure, the isolated nucleic acid molecule is operably linked to a promoter.

In some embodiments of the present disclosure, the promoter is one selected from λ-PL promoter, tac promoter, trp promoter, araBAD promoter, T7 promoter and trc promoter.

According to a fourth aspect of the present disclosure, provided in embodiments is a host cell comprising the construct as described in the third aspect. The host cell for expressing a target gene or a nucleic acid molecule may be a prokaryotic cell. In at least some embodiments, the reverse transcriptase of the present disclosure is expressed by prokaryotic cells, such as Escherichia coli.

According to a fifth aspect of the present disclosure, provided in embodiments is a method for producing a reverse transcriptase as described in the first aspect. The method comprises: culturing a host cell, wherein the host cell is the host cell as described in the fourth aspect, inducing the host cell to express the reverse transcriptase, and isolating the reverse transcriptase.

In some embodiments of the present disclosure, the host cell is Escherichia coli.

According to a sixth aspect of the present disclosure, provided in embodiments is a kit comprising the reverse transcriptase as described in the first aspect. Use of the kit comprising the reverse transcriptase can improve the efficiency of reverse transcription reaction.

In some embodiments of the present disclosure, the kit described above may further have the following technical features.

In some embodiments of the present disclosure, the kit further comprises at least one from one or more nucleotides, one or more DNA polymerases, one or more buffers, one or more primers, and one or more terminators.

In some embodiments of the present disclosure, the terminator is dideoxynucleotide.

According to a seventh aspect of the present disclosure, provided in embodiments is a method for reverse transcription of nucleic acid molecules, comprising: mixing at least one nucleic acid template with at least one reverse transcriptase to obtain a mixture, wherein the reverse transcriptase is the reverse transcriptase as described in the first aspect, and subjecting the mixture to a reverse transcription reaction to obtain a first nucleic acid molecule, wherein the first nucleic acid molecule is completely or partially complementary to the at least one nucleic acid template.

According to an embodiment of the present disclosure, the above-mentioned method for reverse transcription of nucleic acid molecules may further have the following technical features.

In some embodiments of the present disclosure, the first nucleic acid molecule is a cDNA molecule.

In some embodiments of the present disclosure, the nucleic acid template is mRNA.

In some embodiments of the present disclosure, an amount of the nucleic acid template is at least 10 pg.

In some embodiments of the present disclosure, the method further comprises: subjecting the first nucleic acid molecule to a PCR reaction, to obtain a second nucleic acid molecule, wherein the second nucleic acid molecule is completely or partially complementary to the first nucleic acid molecule.

According to an eighth aspect of the present disclosure, provided in embodiments is a method for amplifying nucleic acid molecules, comprising: subjecting at least one nucleic acid template and at least one reverse transcriptase to a first mixing reaction, to obtain a reaction product, wherein the at least one reverse transcriptase is the reverse transcriptase as described in the first aspect, and subjecting the reaction product and at least one DNA polymerase to a second mixing reaction, to obtain an amplified nucleic acid molecule, wherein the amplified nucleic acid molecule is completely or partially complementary to the at least one nucleic acid template. “Mixing reaction” means the reaction between raw materials after the raw materials are mixed.

In some embodiments of the present disclosure, the method for amplifying nucleic acid molecules further comprises: sequencing the amplified nucleic acid molecule to determine a nucleotide sequence of the amplified nucleic acid molecule.

According to a ninth aspect of the present disclosure, provided in embodiments is a method for constructing a cDNA library, comprising: subjecting a biological sample to be tested to RNA extraction, to obtain mRNA of the biological sample to be tested, treating the mRNA of the biological sample to be tested by the method as described in the seventh aspect, to obtain cDNA molecules, and subjecting the cDNA molecules to amplification and library construction to obtain a cDNA library.

In some embodiments of the present disclosure, the above method for constructing a cDNA library may further have the following technical features.

In some embodiments of the present disclosure, the biological sample to be tested is an animal tissue, a plant tissue or bacteria. For example, multiple cells or a single cell in these biological samples can be processed to obtain RNA.

In some embodiments of the present disclosure, a total RNA content in the biological sample to be tested is at least 10 pg.

In some embodiments of the present disclosure, the biological sample to be tested is at least one selected from soil, feces, blood and serum. Biological samples with different sources contain a variety of inhibitors that inhibit the activity of MMLV RT, such as humic acid in soil and feces, hemoglobin in blood, various blood anticoagulants in serum such as heparin and citrate, as well as guanidine and thiocyanic ester, ethanol, formamide, EDTA and plant acid polysaccharides, and the like. Therefore, improving the anti-inhibitor capacity of the enzyme can more effectively expand its application range.

In some embodiments of the present disclosure, a length of obtained cDNA is at least 2000 bp. The reverse transcriptase provided in the present disclosure can be useful in the reverse transcription reaction of large fragments of mRNA, to obtain long fragments of cDNA. According to embodiments of the present disclosure, the length of obtained cDNA may be 500 bp or above, 1000 bp or above, 2000 bp or above, 3000 bp or above, 4000 bp or above, 5000 bp or above, 6000 bp or above, 7000 bp or above, 8000 bp or above, or 9000 bp.

The beneficial effect obtained by this present disclosure is that the reverse transcriptase provided in this present disclosure has good thermal stability, low RNase H activity, and high polymerase activity. The reverse transcriptase when used in the reverse transcription reaction can realize the amplification of complex templates and the full length of cDNA, and improves the amplification efficiency due to the increased reaction tolerance temperature.

### BRIEF DESCRIPTION OF THE DRAWINGS

The above and/or additional aspects and advantages of the present disclosure will become obvious and easy to understand from the description of embodiments in conjunction with the following drawings, in which:

FIG. 1 is a schematic diagram of an M-MLV RT expression vector provided according to an embodiment of the present disclosure.

FIG. 2 is a graph showing screening results of thermal stability of crude enzyme solution of wild-type M-MLV RT and mutants provided according to an embodiment of the present disclosure.

FIG. 3 is a graph showing screening results of thermal stability of pure enzyme solution of wild-type M-MLV RT and mutants provided according to an embodiment of the present disclosure.

FIG. 4 is a graph showing assay results of polymerase activity of crude enzyme solution of wild-type M-MLV RT and mutants provided according to an embodiment of the present disclosure.

FIG. 5 is a graph showing assay results of polymerase activity of pure enzyme solution of wild-type M-MLV RT and mutants provided according to an embodiment of the present disclosure.

FIG. 6 is a graph showing real-time fluorescence curve of wild-type M-MLV RT and mutants provided according to an embodiment of the present disclosure.

FIG. 7 is a graph showing assay results of screening RNase H activity of crude enzyme solution of wild-type M-MLV RT and mutants provided according to an embodiment of the present disclosure.

FIG. 8 is a graph showing assay results of screening RNase H activity of pure enzyme solution of wild-type M-MLV RT and mutants provided according to an embodiment of the present disclosure.

FIG. 9 is a graph showing results of length and yield of cDNA synthesized by wild-type M-MLV RT and mutants according to an embodiment of the present disclosure.

FIG. 10 is a graph showing results of sensitivity of different reverse transcriptases according to an embodiment of the present disclosure.

FIG. 11 is a graph showing cDNA yield and fragment distribution of M-MLV RT mutants in conventional RNA-seq according to an embodiment of the present disclosure.

FIG. 12 is a graph showing a result of M-MLV RT single cell plus C-tail function according to an embodiment of the present disclosure.

FIG. 13 is a graph showing cDNA yield and fragment distribution of M-MLV RT mutants provided according to an embodiment of the present disclosure.

### DETAILED DESCRIPTION

Embodiments of the present disclosure are described in detail below. Examples of the embodiments are shown in the drawings, in which the same or similar reference numerals indicate the same or similar elements or elements with the same or similar functions. The embodiments described below with reference to the drawings are exemplary, and are intended to explain the present disclosure, which should not be construed as limiting the present disclosure.

In order to facilitate understanding, the terms herein are explained and described below. Those skilled in the art should understand that these explanations and descriptions should not be construed as limiting the protection scope of the present disclosure.

As used herein, the term “reverse transcriptase” refers to a protein, polypeptide or polypeptide fragment that exhibits reverse transcriptase activity.

The terms “reverse transcriptase activity”, “reverse transcription activity” or “reverse transcription” refer to the ability of synthesizing DNA strands in the presence of enzymes and RNA as a template.

The terms “mutation”, “mutant”, “mutant type” or the like means having one or more mutations compared to a wild-type DNA sequence or a wild-type amino acid sequence. Of course, this mutation can occur at a nucleic acid level or at an amino acid level.

In the present disclosure, when a mutation site is referred to, it is usually expressed in the art as “abbreviation of amino acid before mutation+site+abbreviation of amino acid after mutation”, such as “R450H”, where “R” represents the amino acid before mutation, “450” represents the corresponding mutation site, and “H” represents the amino acid after mutation. The “R” and “H” are both single-letter abbreviations commonly used in the art to represent amino acids. When a mutation combination is referred to, a “-” is used to connect two mutations. For example, a mutation site “T306K-D583G” represents that the 306th amino acid and the 583th amino acid are both mutated compared to a wild type.

The present disclosure provides reverse transcriptases and a composition containing these reverse transcriptases. The present disclosure provides a composition including one or more (for example, two, three, four, eight, ten, fifteen or the like) polypeptides having present reverse transcriptase activity and useful in reverse transcription of nucleic acid molecules. In addition to these reverse transcriptases, the composition may also include one or more nucleotides, one or more buffers, and one or more DNA polymerases. The composition of the present disclosure may also include one or more oligonucleotide primers.

The reverse transcriptase provided in the present disclosure includes at least one of mutations from R450H, E286K-E302K-W313F-D524A-D583G, T306K-D583G, E562K-D583N, W313F-D524G-D583N, T306K-D524A, E302K-D524A, E302K-L435R-D524A, L435G-D524A, E302K-L435R-D524A-E562Q, E302K-L435G-D524A, D524G-R450H, W313F-D524A, W313F-E562K-D583N, D583N-E562Q, E286K-E302K-W313F-T330P-D524A-D583G, D524G-D583N-R450H, E302R-W313F-L435G and W313F-L435G, compared to the amino acid sequence of SEQ ID NO: 2.

In some embodiments of the present disclosure, the reverse transcriptase has an R450H mutation compared to the amino acid sequence of SEQ ID NO: 2.

In some embodiments of the present disclosure, the reverse transcriptase has E286K-E302K-W313F-D524A-D583G mutations compared to the amino acid sequence of SEQ ID NO: 2.

In some embodiments of the present disclosure, the reverse transcriptase has T306K-D583G mutations compared to the amino acid sequence of SEQ ID NO: 2.

In some embodiments of the present disclosure, the reverse transcriptase has E562K-D583N mutations compared to the amino acid sequence of SEQ ID NO: 2.

In some embodiments of the present disclosure, the reverse transcriptase has W313F-D524G-D583N mutations compared to the amino acid sequence of SEQ ID NO: 2.

In some embodiments of the present disclosure, the reverse transcriptase has T306K-D524A mutations compared to the amino acid sequence of SEQ ID NO: 2.

In some embodiments of the present disclosure, the reverse transcriptase has E302K-D524A mutations compared to the amino acid sequence of SEQ ID NO: 2.

In some embodiments of the present disclosure, the reverse transcriptase has E302K-L435R-D524A mutations compared to the amino acid sequence of SEQ ID NO: 2.

In some embodiments of the present disclosure, the reverse transcriptase has L435G-D524A mutations compared to the amino acid sequence of SEQ ID NO: 2.

In some embodiments of the present disclosure, the reverse transcriptase has E302K-L435R-D524A-E562Q mutations compared to the amino acid sequence of SEQ ID NO: 2.

In some embodiments of the present disclosure, the reverse transcriptase has E302K-L435G-D524A mutations compared to the amino acid sequence of SEQ ID NO: 2.

In some embodiments of the present disclosure, the reverse transcriptase has D524G-R450H mutations compared to the amino acid sequence of SEQ ID NO: 2.

In some embodiments of the present disclosure, the reverse transcriptase has W313F-D524A mutations compared to the amino acid sequence of SEQ ID NO: 2.

In some embodiments of the present disclosure, the reverse transcriptase has W313F-E562K-D583N mutations compared to the amino acid sequence of SEQ ID NO: 2.

In some embodiments of the present disclosure, the reverse transcriptase has D583N-E562Q mutations compared to the amino acid sequence of SEQ ID NO: 2.

In some embodiments of the present disclosure, the reverse transcriptase has E286K-E302K-W313F-T330P-D524A-D583G mutations compared to the amino acid sequence of SEQ ID NO: 2.

In some embodiments of the present disclosure, the reverse transcriptase has D524G-D583N-R450H mutations compared to the amino acid sequence of SEQ ID NO: 2.

In some embodiments of the present disclosure, the reverse transcriptase has E302R-W313F-L435G mutations compared to the amino acid sequence of SEQ ID NO: 2.

In some embodiments of the present disclosure, the reverse transcriptase has W313F-L435G mutations compared to the amino acid sequence of SEQ ID NO: 2.

The reverse transcriptase provided in the present disclosure is resistant to enzyme inhibitors presented in a biological sample. The biological sample may be, for example, blood, feces, animal tissues, plant tissues, bacteria, sweat, tears, dust, saliva, urine, bile and the like. These enzyme inhibitors may be humic acid, heparin, ethanol, bile salts, fulvic acid, metal ions, sodium lauryl sulfate, EDTA, guanidine salts, formamide, sodium pyrophosphate and spermidine. When these biological samples or samples containing these inhibitors are subjected to reverse transcription, the reverse transcriptase provided in the present disclosure can exhibit at least 10% reverse transcriptase activity. More specifically, in the presence of inhibitors, the reverse transcriptase provided in the present disclosure can exhibit 10%, 20%, 30%, 40%, 50%, 60%, 70%, 80% or even 90% of reverse transcriptase activity, compared to samples without inhibitors.

The present disclosure also provides a kit. The kit provided in the present disclosure can be used to generate and amplify nucleic acid molecules (single-stranded or double-stranded) or be used for sequencing. The kit provided in this present disclosure includes a loadable carrier, such as a box or a hard box and so on. These loadable carriers include one or more containers, such as a vial, a tube, etc. These containers can be provided with one or more reverse transcriptases provided in the present disclosure. Besides, in addition to reverse transcriptase, one or more DNA polymerases, one or more buffers suitable for nucleic acid synthesis, and one or more nucleotides can also be disposed in same or different containers.

The technical solution of the present disclosure will be explained below in conjunction with examples. Those skilled in the art will understand that the following examples are only used to illustrate the present disclosure, and should not be regarded as limiting the scope of the present disclosure. Where specific techniques or conditions are not indicated in the examples, the procedures are carried out in accordance with the techniques or conditions described in the literature in the field or in accordance with the product specification. The reagents or instruments used without the manufacturer's indication are all conventional products that can be purchased commercially.

### Example 1 Construction of Expression Vectors of Wild-Type M-MLV RT and its Mutants

1. Construction of Expression Vector of Wild-Type M-MLV RT

According to the NCBI database, the nucleic acid sequence of reverse transcriptase derived from Moloney murine leukaemia virus (M-MLV) was obtained. Despite existing codons that are difficult for Escherichia coli to recognize in the obtained nucleic acid sequence, such codons in the nucleic acid sequence that are difficult for E. coli to recognize are changed to codons commonly used in E. coli, which makes the gene more conducive to expression in E. coli, and thus obtaining optimized nucleic acid sequence. After which, the optimized nucleic acid sequence was introduced into an expression plasmid to obtain the expression vector.

Among them, the nucleic acid sequence of wild-type M-MLV RT (optimized) shown in SEQ ID NO: 1 is as follows:

ATGCTGAACATCGAGGACGAACACCGTCTGCACGAGACCAGCAAGGAACCG
GACGTGAGCCTGGGTAGCACCTGGCTGAGCGATTTCCCGCAGGCGTGGGCG
GAGACCGGTGGCATGGGTCTGGCGGTGCGTCAAGCGCCGCTGATCATTCCG
CTGAAGGCGACCAGCACCCCGGTTAGCATCAAACAGTACCCGATGAGCCAA
GAAGCGCGTCTGGGTATCAAACCGCACATTCAGCGTCTGCTGGACCAAGGC
ATTCTGGTTCCGTGCCAAAGCCCGTGGAACACCCCGCTGCTGCCGGTGAAG
AAACCGGGCACCAACGACTATCGTCCGGTTCAGGATCTGCGTGAGGTGAAC
AAGCGTGTTGAAGATATCCACCCGACCGTGCCGAACCCGTACAACCTGCTG
AGCGGTCTGCCGCCGAGCCATCAGTGGTATACCGTTCTGGACCTGAAAGAT
GCGTTCTTTTGCCTGCGTCTGCATCCGACCAGCCAGCCGCTGTTTGCGTTT
GAGTGGCGTGACCCGGAAATGGGTATTAGCGGTCAGCTGACCTGGACCCGT
CTGCCGCAAGGCTTCAAGAACAGCCCGACCCTGTTTGACGAGGCGCTGCAC
CGTGACCTGGCGGATTTTCGTATCCAGCACCCGGATCTGATTCTGCTGCAA
TACGTGGACGATCTGCTGCTGGCGGCGACCAGCGAACTGGATTGCCAGCAA
GGTACCCGTGCGCTGCTGCAGACCCTGGGTAACCTGGGCTATCGTGCGAGC
GCGAAGAAAGCGCAAATCTGCCAGAAGCAAGTGAAATACCTGGGTTATCTG
CTGAAAGAGGGTCAGCGTTGGCTGACCGAGGCGCGTAAGGAAACCGTTATG
GGTCAGCCGACCCCGAAAACCCCGCGTCAACTGCGTGAGTTCCTGGGTACC
GCGGGCTTTTGCCGTCTGTGGATTCCGGGTTTTGCGGAAATGGCGGCGCCG
CTGTACCCGCTGACCAAAACCGGTACCCTGTTTAACTGGGGCCCGGACCAG
CAAAAGGCGTATCAGGAAATTAAACAAGCGCTGCTGACCGCGCCGGCGCTG
GGTCTGCCGGACCTGACCAAGCCGTTCGAGCTGTTTGTGGATGAAAAGCAG
GGTTACGCGAAAGGCGTTCTGACCCAAAAACTGGGTCCGTGGCGTCGTCCG
GTGGCGTATCTGAGCAAGAAACTGGACCCGGTTGCGGCGGGTTGGCCGCCA
TGCCTGCGTATGGTGGCGGCGATCGCGGTTCTGACCAAGGATGCGGGTAAA
CTGACCATGGGTCAGCCGCTGGTGATTCTGGCGCCGCACGCGGTGGAGGCG
CTGGTTAAACAACCGCCGGATCGTTGGCTGAGCAACGCGCGTATGACCCAC
TACCAGGCGCTGCTGCTGGACACCGATCGTGTTCAATTTGGTCCGGTGGTT
GCGCTGAACCCGGCGACCCTGCTGCCGCTGCCGGAGGAAGGTCTGCAGCAC
AACTGCCTGGACATTCTGGCGGAGGCGCATGGTACCCGTCCGGACCTGACC
GATCAACCGCTGCCGGACGCGGATCACACCTGGTATACCGATGGTAGCAGC
CTGCTGCAGGAAGGTCAGCGTAAAGCGGGTGCGGCGGTGACCACCGAGACC
GAAGTTATCTGGGCGAAGGCGCTGCCGGCGGGTACCAGCGCGCAGCGTGCG
GAGCTGATTGCGCTGACCCAAGCGCTGAAGATGGCGGAAGGCAAGAAACTG
AACGTTTACACCGACAGCCGTTATGCGTTCGCGACCGCGCACATCCACGGC
GAGATTTACCGTCGTCGTGGTCTGCTGACCAGCGAGGGCAAGGAAATCAAG
AACAAGGATGAAATCCTGGCGCTGCTGAAGGCGCTGTTTCTGCCGAAACGT
CTGAGCATCATTCACTGCCCGGGTCACCAGAAAGGTCACAGCGCGGAGGCG
CGTGGTAACCGTATGGCGGACCAAGCGGCGCGTAAAGCGGCGATCACCGAA
ACCCCGGATACCAGCACCCTGCTGATT

The amino acid sequence of wild-type M-MLV RT shown in SEQ ID NO: 2 is as follows:

MLNIEDEHRLHETSKEPDVSLGSTWLSDFPQAWAETGGMGLAVRQAPLIIP
LKATSTPVSIKQYPMSQEARLGIKPHIQRLLDQGILVPCQSPWNTPLLPVK
KPGTNDYRPVQDLREVNKRVEDIHPTVPNPYNLLSGLPPSHQWYTVLDLKD
AFFCLRLHPTSQPLFAFEWRDPEMGISGQLTWTRLPQGFKNSPTLFDEALH
RDLADFRIQHPDLILLQYVDDLLLAATSELDCQQGTRALLQTLGNLGYRAS
AKKAQICQKQVKYLGYLLKEGQRWLTEARKETVMGQPTPKTPRQLREFLGT
AGFCRLW1PGFAEMAAPLYPLTKTGTLFNWGPDQQKAYQEIKQALLTAPAL
GLPDLTKPFELFVDEKQGYAKGVLTQKLGPWRRPVAYLSKKLDPVAAGWPP
CLRMVAAIAVLTKDAGKLTMGQPLVILAPHAVEALVKQPPDRWLSNARMTH
YQALLLDTDRVQFGPVVALNPATLLPLPEEGLQHNCLDILAEAHGTRPDLT
DQPLPDADHTWYTDGSSLLQEGQRKAGAAVTTETEVIWAKALPAGTSAQRA
ELIALTQALKMAEGKKLNVYTDSRYAFATAHIHGEIYRRRGLLTSEGKEIK
NKDEILALLKALFLPKRLSIIHCPGHQKGHSAEARGNRMADQAARKAAITE
TPDTSTLLI

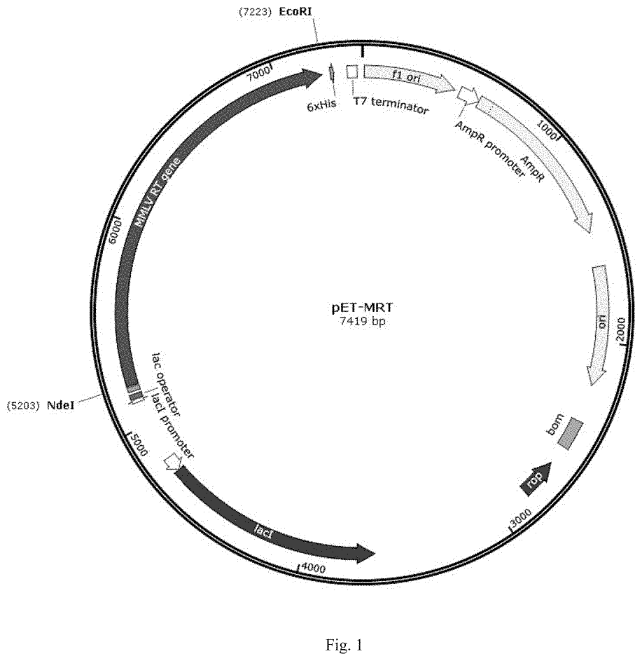

The wild-type m-mlv rt gene sequence (SEQ ID NO: 1) was inserted between NdeI and EcoRI restriction sites of an expression plasmid pET22b(+). The expression vector has 6 His at the C-terminus of the m-mlv rt sequence, to facilitate protein purification. The expression vector was named pET-MRT, as shown in FIG. 1.

2. Construction of Expression Vectors of M-MLV RT Mutants

For mutation sites that may be beneficial to improve the thermal stability of reverse transcriptase and reduce the RNase H activity, forward and reverse primer pairs for the mutation sites were designed. Site-directed mutation PCR was performed by using pET-MRT as a template and Pfu DNA polymerase (EP0501, Thermo Fisher), to obtain the corresponding expression vectors of M-MLV RT mutants. Among them, different forward and reverse primers can be designed for different mutation sites for performing site-directed mutation. Methods are as below:

(1) Site-directed mutation was performed according to the following reaction system and reaction conditions.

TABLE 1
PCR reaction system for constructing expression
vectors of M-MLV RT mutants

| Reaction component | Volume (μl) |
|---|---|
| 10× Pfu buffer (with MgSO₄) | 2.5 |
| 2.5 mM dNTPs | 2 |
| 10 μM forward primer | 0.7 |
| 10 μM reverse primer | 0.7 |
| pfu DNA polymerase | 0.5 |
| 50 ng/μl template (pET-MRT) | 1 |
| H₂O | 17.6 |

TABLE 2
PCR reaction condition for constructing expression vectors of
M-MLV RT mutants Reaction condition

| 95° C., 5 min | | |
|---|---|---|
| 95° C., 30 s | | |
| 53° C., 30 s | {close oversize brace} | 19 cycles |
| 68° C., 8 min | | |

68° C., 10 min
4° C., ∞

(2) After the reaction, 1 μl DpnI was added for digestion at 37° C. for 2 hours;

(3) 5 μl of digested product was taken to transform E. coli DH5α competent cells;

(4) a single clone was picked from the plate and cultured in LB medium containing ampicillin antibiotics at 37° C. with shaking at 200 rpm;

(5) the plasmid was extracted, sequenced and comparatively analyzed to obtain the clone with correct mutation.

The constructed mutants are as follows.

TABLE 3
M-MLV RT reverse transcriptase mutation information

| No. | Mutation site |
|---|---|
| RT-1 | E302K |
| RT-2 | L435G |
| RT-3 | D524A |
| RT-4 | E562Q |
| RT-5 | D583G |
| RT-6 | D524N |
| RT-7 | N454R |
| RT-8 | E286K |
| RT-9 | W313F |
| RT-10 | D583N |
| RT-11 | D524G |
| RT-12 | R450H |
| RT-13 | T330P |
| RT-14 | E562K |
| RT-15 | T306K |
| RT-16 | E302R |
| RT-17 | E302K-L435R-D524A-E |
| | 562Q |
| RT-18 | E302K-L435R-D524A-D |
| | 583G |
| RT-19 | L435R-D524A-D583G |
| RT-20 | D524N-D583G |
| RT-21 | D524N-N454R |
| RT-22 | E286K-E302K-W313F- |
| | D524A-D583G |
| RT-23 | W313F-D583N |
| RT-24 | D524G-D583N-R450H |
| RT-25 | E286K-E302K-W313F-T330P-D524A- |
| | D583G |
| RT-26 | W313F-D524G |
| RT-27 | W313F-D524G-D583N |
| RT-28 | W313F-E562K-D583N |
| RT-29 | T306K-D524A |
| RT-30 | T306K-D583G |
| RT-31 | W313F-D524A |
| RT-32 | D583N-D524G |
| RT-33 | D583N-E562Q |
| RT-34 | D524G-E562Q |
| RT-35 | E562K-D583N |
| RT-36 | E302R-W313F-L435G |
| RT-37 | W313F-L435G |
| RT-38 | E302R-W313F |
| RT-39 | D524G-R450H |
| RT-40 | L435G-D524A |
| RT-41 | E302K-L435G-D524A |
| RT-42 | E286K-E302K-D524A |
| RT-43 | E302K-D524A |
| RT-44 | E302K-L435R-D524A |

### Example 2 Expression and Purification of Wild-Type M-MLV RT Reverse Transcriptase and its Mutants

1. Wild-type M-MLV RT reverse transcriptase and its mutants were induced and expressed in a small amount and purified to obtain crude enzymes.

Wild-type M-MLV RT reverse transcriptase and its mutants were all expressed by the promoter of pET22b, and 6 His tags were fused to the C-terminus, which were used for Ni column affinity purification during the purification process to obtain the corresponding crude enzyme solution. Methods are as below:

(1) The wild-type and mutant plasmids were transformed into BL21 competent cells (purchased from TransGen Biotech Co., Ltd.);

(2) a single colony was picked and cultured in 10 ml of LB medium containing ampicillin resistance (100 μg/ml) at 37° C. and 200 rpm/min of shaking until OD600≈0.6;

(3) the inducer IPTG (a final concentration of 0.5 mM) was added, and induced overnight at 18° C. and 200 rpm/min;

(4) the culture was centrifuged at 12000 rpm/min for 5 minutes, and the induced bacterial cell precipitate was collected;

(5) the induced bacterial cell precipitate was resuspended with M-MLV RT resuspension solution (containing 20 mM Tris-HCl, 500 mM NaCl, 20 mM Imidazole, 5% Glycerol, pH 7.5) and incubated at 25° C., and 1% 10 mg/ml Lysozyme, 1% PMSF and 0.5% TritonX-100 were added. Bacterial cells were broken by ultrasound under ice-water bath conditions, and the ultrasonic conditions are that: amplitude transformer bar diameter is φ10, power is 35%, and ultrasonic treatment is 2s, intermittence is 3s and then ultrasonic treatment is 5 mins;

(6) the broken bacteria solution was centrifuged at 12000 rpm and 4° C. for 10 mins and the supernatant was collected.

The supernatant of MMLV RT crude enzyme prepared in the previous step was subjected to Ni column affinity purification. The main steps are that combining filler with the crude enzyme solution by incubation; resuspension to wash proteins unbound to Ni column; and eluting target protein at 25° C. by using the eluent (20 mM Tris-HCl, 500 mM NaCl, 260 mM Imidazole, 5% Glycerol, pH 7.5) to obtain the crude enzyme solution.

The concentration of the target protein obtained after purification was determined at A280 and adjusted to a same concentration for subsequent screening experiments.

2. Wild-type M-MLV RT reverse transcriptase and its mutants were induced and expressed in a large amount and purified to obtain pure enzymes.

Wild-type M-MLV RT reverse transcriptase and its mutants were all expressed by the promoter of pET22b, and 6 His tags were fused to the C-terminus, which were used for Ni column affinity purification during the purification process to obtain the corresponding pure enzyme solution.

(1) The wild-type and mutant plasmids were transformed into BL21 competent cells (purchased from TransGen Biotech Co., Ltd.);

(2) a single colony was picked and cultured in 5 ml of LB medium containing ampicillin resistance (100 μg/ml) overnight at 37° C. and 200 rpm/min, which was diluted at a ratio of 1:100 the next day and transferred to 1500 ml of fresh LB medium containing ampicillin resistance (100 μg/ml), cultured at 37° C. and 200 rpm/min of shaking, until OD600≈0.6;

(3) the inducer IPTG (a final concentration of 0.5 mM) was added, and induced overnight at 18° C. and 200 rpm/min;

(4) the culture was centrifuged at 8000 rpm/min for 10 minutes, and the induced bacterial cell precipitate was collected;

(5) the induced bacterial cell precipitate was resuspended with M-MLV RT resuspension solution (containing 20 mM Tris-HCl, 500 mM NaCl, 20 mM Imidazole, 5% Glycerol, pH 7.5) and incubated at 25° C., and 1% 10 mg/ml Lysozyme, 1% PMSF and 0.5% TritonX-100 were added. Bacterial cells were broken by ultrasound under ice-water bath conditions, and the ultrasonic conditions are that amplitude transformer bar diameter is φ10, power is 35%, and ultrasonic treatment is 2s, intermittence is 3s and then ultrasonic treatment is 5 mins;

(6) the broken bacteria solution was centrifuged at 12000 rpm and 4° C. for 30 mins and the supernatant was collected.

The sample prepared in the previous step was subjected to affinity purification by the AKTA protein purification system, and the sample obtained via affinity purification was diluted in 3.33 times with M-MLV RT diluent (20 mM Tris-HCl, 5% Glycerol, pH7.5), followed by anion exchange chromatography to obtain the purified target protein, which is the pure enzyme solution.

The target protein obtained after purification was dialyzed and stored for subsequent assays and analysis.

### Example 3 Screening and Analysis of Thermal Stability of Wild-Type M-MLV RT Reverse Transcriptase and its Mutants

M-MLV RT is a normal temperature enzyme. The T50 (the temperature at which the enzyme activity decreases to 50% of the initial enzyme activity in 10 minutes) of wild-type M-MLV RT is 44° C. when the substrate is not present and is 47° C. when the substrate is present. In the present disclosure, the wild-type M-MLV RT and its mutants were tested for thermal stability through a kit. At the same time, by comparing polymerized product amounts of the mutants at different temperatures and the activity retention rate of the wild-type M-MLV RT and its mutants, the mutants with more stable thermal-stability than the wild-type were screened.

The thermal stability of the crude enzyme solution and pure enzyme solution of the wild-type M-MLV RT reverse transcriptase and its mutants were measured. The test kit (Protein Thermal Shift™ Dye Kit purchased from Thermal) is used in the thermal stability detection. The specific detection principle is, as the temperature rises, the protein structure changes, the hydrophobic domain is exposed, which is combined by the fluorescent dye to produce fluorescence. The change between the temperature and the fluorescence value (Melt Curve) was detected in real time by the qPCR instrument, and Tm value of the wild type M-MLV RT reverse transcriptase and its mutants were compared to determine the thermal stability.

A 96-well plate was used to prepare a reaction system according to the kit operation mentioned above. The specific reaction system is as follows.

TABLE 4
reaction system for screening thermal stability of
M-MLV RT reverse transcriptase

| Reaction components | Volume (μl) |
|---|---|
| Protein Thermal Shift ™ Buffer | 5 |
| M-MLV RT reverse transcriptase enzyme | 12.5 |
| solution (0.3 m/ml) | |
| Diluted Protein Thermal Shift ™ Dye (8×) | 2.5 |

Notes: the M-MLV RT reverse transcriptase enzyme solution (0.3 mg/ml) in the above table refers to an enzyme solution to be tested with a concentration of 0.3 mg/ml obtained by diluting the purified enzyme solution obtained in Example 2 by a certain multiple times, the Dye means the dye (1000×) in the kit is diluted to 8× with sterile water, and a 96-well plate is used for detection.

After addition of the sample, Melt Curve was prepared by the StepOne™ qPCR instrument. The specific Melt curve reaction conditions are completely set according to the kit instruction.

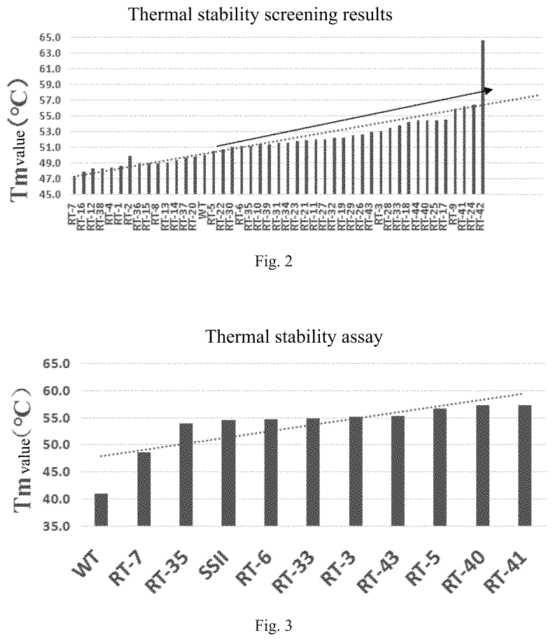

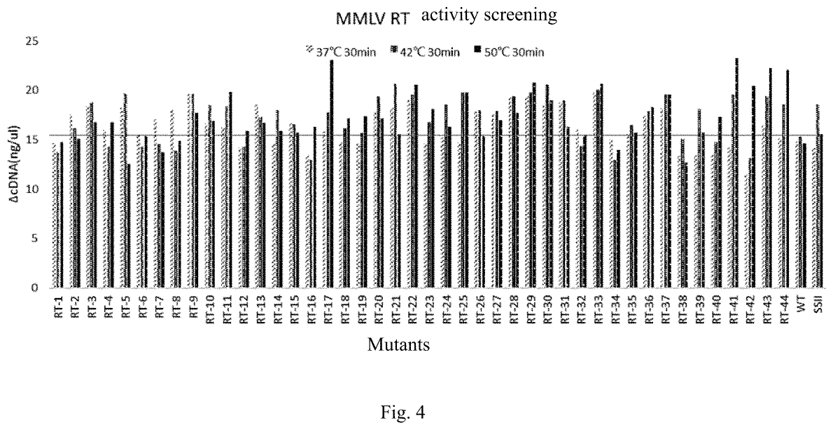

The specific Tm values of the wild-type M-MLV RT reverse transcriptase and its mutants were analyzed by the Protein Thermal Shift™ software v1.0. The results are shown in Table 5 and FIGS. 2 and 3.

TABLE 5
Screening results of thermal stability of crude enzyme solution of
wild-type M-MLV RT reverse transcriptase and mutants

| | Tm value | | Tm value | | Tm value |
|---|---|---|---|---|---|
| No. | (° C.) | No. | (° C.) | No. | (° C.) |
| RT-7 | 47.2 | RT-5 | 50.6 | RT-29 | 52.5 |
| RT-16 | 47.9 | RT-22 | 50.7 | RT-26 | 52.7 |
| RT-12 | 48.3 | RT-30 | 51.1 | RT-43 | 53.0 |
| RT-38 | 48.3 | RT-6 | 51.1 | RT-3 | 53.1 |
| RT-4 | 48.5 | RT-35 | 51.2 | RT-28 | 53.5 |
| RT-1 | 48.7 | RT-10 | 51.4 | RT-33 | 53.8 |
| RT-2 | 49.9 | RT-39 | 51.4 | RT-18 | 54.2 |
| RT-36 | 48.9 | RT-31 | 51.6 | RT-44 | 54.4 |
| RT-15 | 48.9 | RT-34 | 51.6 | RT-40 | 54.4 |
| RT-8 | 48.9 | RT-23 | 51.8 | RT-25 | 54.5 |
| RT-13 | 49.0 | RT-21 | 52.0 | RT-17 | 54.6 |
| RT-14 | 49.3 | RT-11 | 52.0 | RT-9 | 55.9 |
| RT-37 | 49.7 | RT-27 | 52.0 | RT-41 | 56.2 |
| RT-20 | 49.8 | RT-32 | 52.2 | RT-24 | 56.4 |
| WT | 50.0 | RT-19 | 52.3 | RT-42 | 64.7 |

Among them, FIG. 2 shows the measurement results of thermal stability of crude enzyme solution of wild-type M-MLV RT reverse transcriptase and mutants. FIG. 3 shows the measurement results of thermal stability of pure enzyme solution of wild-type M-MLV RT reverse transcriptase and mutants. The results shown in Table 5 correspond to the results shown in FIG. 2. The black arrow area in FIG. 2 represents the improvement of thermal stability of each test sample. Integrating the thermal stability detection results of the crude enzyme solution and the pure enzyme solution, it is found that the thermal stability detection results of individual mutants in the crude enzyme solution are different from that in the pure enzyme solution. Without being limited by theory, the difference may be caused by the different purity of enzyme solution. Because the purity of crude enzyme solution is not high, some mutants with poor results can be removed with the aid of thermal stability detection results.

### Example 4 Assay and Analysis of Polymerization Activity of Wild-Type M-MLV RT Reverse Transcriptase and its Mutants

M-MLV RT reverse transcriptase is a normal temperature enzyme, and its polymerization activity will decrease as the temperature rises. Therefore, at a same reaction temperature, a mutant with better enzyme activity can be screened by comparing the polymerization product amount of the wild-type M-MLV RT and mutants.

A poly(rA): (dT) hybrid chain was generated via polymerization reaction by reverse transcriptase, poly(rA) as a template and oligo(dT) as a primer. The polymerization reaction was carried out under different reaction temperature conditions, and the product concentration was detected by Qubit dsDNA HS kit (Invitrogen). By comparing the polymerization product amount of the wild-type M-MLV RT reverse transcriptase and its mutants, mutants with mutation combination and single-point mutants, the mutants with better enzyme activity were screened.

TABLE 6
Polymerization reaction system

| Reagent | Volume /ul |
|---|---|
| DEPC H₂O | — |
| 5× RT reaction Buffer (with DTT) | 4.0 |
| 10 mM dTTP | 1.0 |
| 10 uM oligo(dT) | 3.0 |
| 40 U/ul RI | 1.0 |
| Poly(rA) | Final 500 ng |
| RT-mutants/H2O | Final 0.6 ug |
| Vₜₒₜₐₗ | 20 ul |

The polymerization reaction was conducted at each of 37° C., 42° C. and 50° C. for 30 minutes. 1 ul 0.5M EDTA was used to stop the reaction. The obtained product concentration is shown in FIGS. 4 and 5, and the polymerase activity ratio of mutants with WT is shown in Table 7.

TABLE 7
polymerase activity of crude enzyme solution of M-MLV RT mutants

| No. | 42° C. 30 min | 50° C. 30 min |
|---|---|---|
| RT-1 | 0.88 | 1.14 |
| RT-2 | 0.87 | 0.97 |
| RT-3 | 0.96 | 1.03 |
| RT-4 | 0.84 | 1.19 |
| RT-5 | 1.01 | 0.78 |
| RT-6 | 0.87 | 1.13 |
| RT-7 | 0.80 | 0.91 |
| RT-8 | 0.72 | 0.93 |
| RT-9 | 0.94 | 1.02 |
| RT-10 | 1.04 | 1.15 |
| RT-11 | 1.06 | 1.38 |
| RT-12 | 0.94 | 1.26 |
| RT-13 | 0.88 | 1.02 |
| RT-14 | 1.15 | 1.23 |
| RT-15 | 0.94 | 1.06 |
| RT-16 | 0.91 | 1.37 |
| RT-17 | 1.05 | 1.65 |
| RT-18 | 1.03 | 1.32 |
| RT-19 | 1.01 | 1.34 |
| RT-20 | 1.02 | 1.09 |
| RT-21 | 1.06 | 0.97 |
| RT-22 | 0.97 | 1.22 |
| RT-23 | 1.08 | 1.40 |
| RT-24 | 1.14 | 1.21 |
| RT-25 | 1.26 | 1.52 |
| RT-26 | 0.95 | 0.97 |
| RT-27 | 0.95 | 1.09 |
| RT-28 | 0.95 | 1.04 |
| RT-29 | 0.96 | 1.21 |
| RT-30 | 1.05 | 1.16 |
| RT-31 | 0.95 | 0.98 |
| RT-32 | 0.84 | 1.09 |
| RT-33 | 0.96 | 1.18 |
| RT-34 | 0.81 | 1.05 |
| RT-35 | 1.00 | 1.14 |
| RT-36 | 0.96 | 1.18 |
| RT-37 | 1.01 | 1.22 |
| RT-38 | 1.06 | 1.07 |
| RT-39 | 1.26 | 1.33 |
| RT-40 | 1.02 | 1.44 |
| RT-41 | 1.29 | 1.85 |
| RT-42 | 1.08 | 2.02 |
| RT-43 | 1.11 | 1.53 |
| RT-44 | 1.15 | 1.65 |
| WT | 1 | 1 |
| SSII | 1.16 | 1.24 |

FIG. 4 shows the polymerase activity of crude enzyme solution of M-MLV RT reverse transcriptase and its mutants at different temperatures. At the same time, Table 7 shows the product concentration of crude enzyme solution of M-MLV RT reverse transcriptase and its mutants at 42° C. and 50° C. FIG. 5 shows the polymerase activity results of pure enzyme solution of M-MLV RT reverse transcriptase and its some mutants. At the same time, Table 8 shows the product concentration of pure enzyme solution of M-MLV RT reverse transcriptase and its some mutants. The product concentration of the crude enzyme solution at different temperatures shown in Table 7 may have some deviations. Without being limited by theory, these deviations may be caused by the low purity of the crude enzyme solution and the presence of impurities in the crude enzyme solution. The results of crude enzyme solution can be used as an important reference for the characterization of pure enzyme solution.

TABLE 8
polymerase activity of pure enzyme solution of M-MLV RT mutants

| No. | ΔcDNA, ng/ul |
|---|---|
| RT-1 | 12.01 |
| RT-3 | 25.74 |
| RT-5 | 23.94 |
| RT-6 | 17.34 |
| RT-17 | 18.2 |
| RT-25 | 22.74 |
| RT-28 | 17.11 |
| RT-33 | 29.04 |
| RT-35 | 21.24 |
| RT-40 | 20.74 |
| RT-41 | 21.14 |
| RT-43 | 19.24 |
| SSII | 22.54 |
| WT | 6.19 |

### Example 5 Screening and Analysis of RNase H Activity of M-MLV RT Reverse Transcriptase and its Mutants

M-MLV RT reverse transcriptase has RNase H activity and can degrade RNA in the DNA/RNA hybrid strand. According to the principle of fluorescence energy resonance transfer, the fluorescence-quenching group pair can usually provide lower background signal and sensitive fluorescence intensity changes when the quenching group is transferred beyond the energy resonance distance of the fluorescent group. When M-MLV RT reverse transcriptase has RNase H activity, it would degrade the RNA strand (the quenching group BHQ2 is present at the 3′ end) in the hybrid strand, which would cause fluorescence value of 5′ fluorescent group cy3 in the DNA single strand of the hybrid strand increased significantly. Therefore, the mutants with fluorescence value lower than the wild-type M-MLV RT can be screened out, that is mutants with decreased RNase H activity.

After purification of M-MLV RT reverse transcriptase and its mutants, the pure enzyme solution qualified for quality inspection was obtained and detected for RNase H activity.

The fluorescently labeled substrates used in the activity assay system are single-stranded

DNA: Poly (dT) 30
(SEQ ID NO: 3)
5′-cy3TTTTTTTTTTTTTTTTTTTTTTTTTTTTTT-3′,
single-stranded RNA: Poly(rA) 30
(SEQ ID NO: 4)
5′-AAAAAAAAAAAAAAAAAAAAAAAAAAAAAA-3′-BHQ2.

The length is 30-mer, the 5′ end of the single-stranded DNA has a cy3 fluorescent group, and the 3′ end of the single-stranded RNA has a BHQ2 quenching group. The RNA single strand and the DNA single strand are annealed to form a hybrid strand, and the appropriate excitation wavelength and emission wavelength were determined to be 540 nm and 570 nm respectively according to test by the microplate reader.

Experimental process is: annealing to synthesize DNA/RNA hybrid strand, in which the concentrations of substrates Poly(dT)30 and Poly(rA)30 are each 10 μM, and annealing at 80° C. for 5 mins at a ratio of 1:1, and then naturally cooled to room temperature.

The reaction system for assaying the RNase H activity of M-MLV RT reverse transcriptase and its mutants is shown in Table 8.

TABLE 8
Reaction system for assaying RNase H activity of M-MLV RT
reverse transcriptase enzyme solution

| Reaction component | Volume |
|---|---|
| DNA/RNA hybrid strand | 3 μl |
| 10× RNase H reaction buffer | 2.5 μl |
| M-MLV RT reverse transcriptase | 2 μl |
| enzyme solution (0.3 mg/ml) | |
| sterile water | make up to 20 μl |

Notes: the M-MLV RT reverse transcriptase enzyme solution (0.3 mg/ml) in the above table refers to an enzyme solution to be tested with a concentration of 0.3 mg/ml obtained by diluting the purified enzyme solution obtained in Example 2 by a certain multiple times, the Dye means the dye (1000×) in the kit is diluted to 8× with sterile water, and a 384-well plate (Corning black, clear bottom 384 plates) is used for detection. The sample loading operation is performed on ice quickly.

After the sample was added, it was detected on the BioTek microplate reader at 37° C. The detection program ensures that the setting operation is completed before the sample is added, including the selection of corresponding sample adding hole position in the 384-well plate. The specific setting program is that: start kinetics (vibrating the plate for 30 seconds before testing, recording data once every minute), the total detection time is 30 mins, the excitation wavelength is 540 nm, and the emission wavelength is 570 nm.

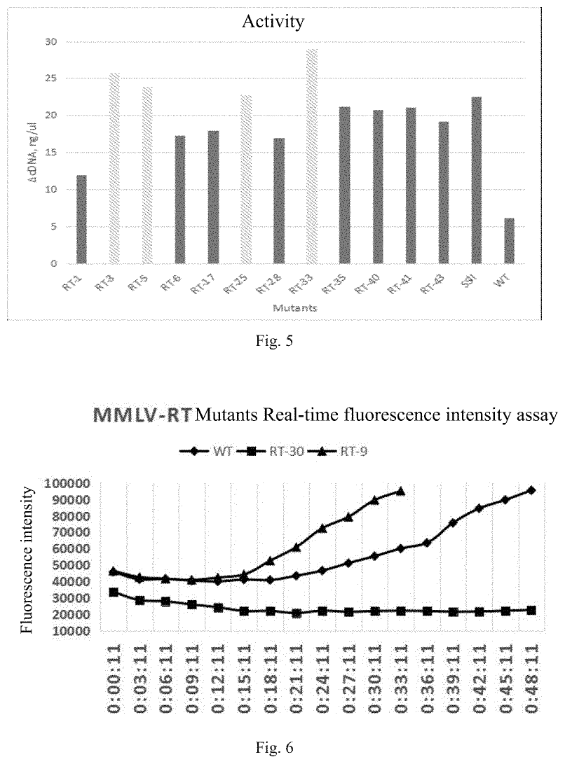

After the detection, the RNase H activity of M-MLV RT and its mutants was analyzed, compared and screened. When the detection is completed, the signal change curve trend graph with the time axis as the abscissa axis and the fluorescence value as the ordinate axis and the corresponding specific data table are derived (see FIGS. 6, 7 and 8).

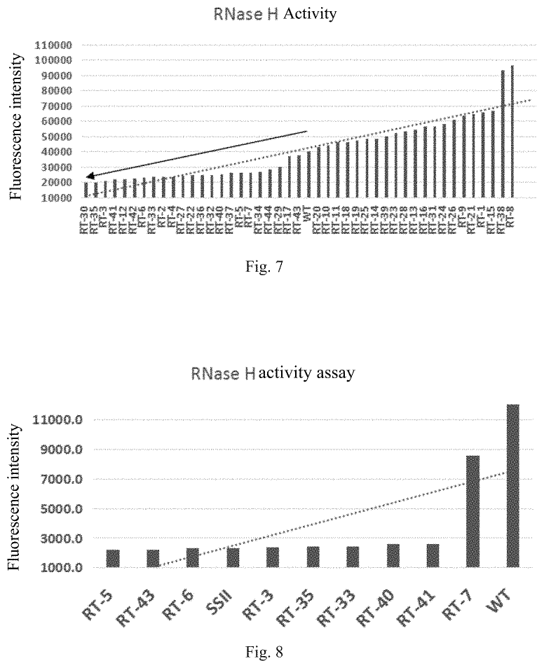

Among them, FIG. 6 shows the real-time fluorescence curve. The middle curve represents the wild-type reverse transcriptase, and the curve below the middle curve represents the mutant has a lower RNase H activity than that of the wild-type. FIG. 7 is a graph showing the screening results of RNase H activity of crude enzyme solution. The black arrow area represents that the RNase H activity of the mutants is decreased compared to the wild-type reverse transcriptase. FIG. 8 is a graph showing the verification results of RNase H activity of pure enzyme solution.

From Examples 3 to 5, the enzyme activity of mutants was verified through different experiments. Based on the verification results of different experiments, only the R450H mutant was retained for the mutants formed by single point mutation; and mutants which have enzyme activity significantly higher than that of wild-type M-MLV reverse transcriptase were retained for the mutants formed by multiple point mutations.

Overall, mutants R450H, E286K-E302K-W313F-D524A-D583G, T306K-D583G, E562K-D583N, W313F-D524G-D583N, T306K-D524A, E302K-D524A, E302K-L435R-D524A, L435G-D524A, E302K-L435R-D524A-E562Q, E302K-L435G-D524A, D524G-R450H, W313F-D524A, W313F-E562K-D583N, D583N-E562Q, E286K-E302K-W313F-T330P-D524A-D583G, D524G-D583N-R450H, E302R-W313F-L435G, W313F-L435G are selected.

### Example 6 Detection and Analysis of cDNA Length of M-MLV RT Reverse Transcriptase and its Mutants

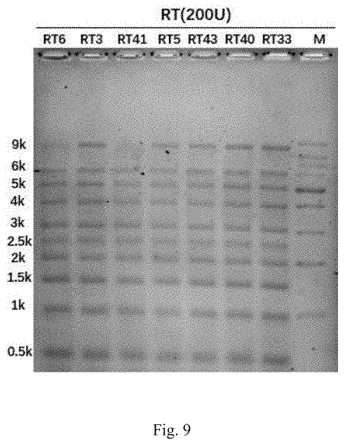

1 ug RNA Marker (0.5 k-9 k) was transcribed by using M-MLV RT reverse transcriptase mutants screened via polymerase activity, RNase H activity and thermal stability (RT3, RT5, RT6, RT33, RT40, RT41, RT43, in which RT3, RT5 and RT6 can be used as a control since reported as existing sites with better effect), along with the commercial ssII. The transcription reaction system and conditions are as shown in Table 9, and the cDNA product was detected by 1% alkaline agarose gel electrophoresis (see FIG. 9).

TABLE 9
transcription reaction system and conditions of reverse transcriptase

| Component | Volume (ul) |
|---|---|
| 1 ug RNA Marker | 1 |
| 50 uM Oligo dT23VN | 1 |
| RNase Free H₂O | Up to 11 ul |

65° C., 5 min

| | |
|---|---|
| 25 mM dNTP | 1 |
| 5× RT buffer | 4 |
| RNase inhibitor | 1 |
| 0.1M DTT | 2 |
| reverse transcriptase | 1 |

42° C., 50 min, 70° C., 10 min

FIG. 9 shows the gel electrophoresis of the cDNA products obtained by using different reverse transcriptases. It can be seen from FIG. 9 that the length of obtained cDNA is between 0.5 and 9 kbp. The results show that RT33, RT40, RT41 and RT43 can all synthesize 9 k of fragments.

### Example 7 Sensitivity Detection and Analysis of M-MLV RT Reverse Transcriptase and its Mutants

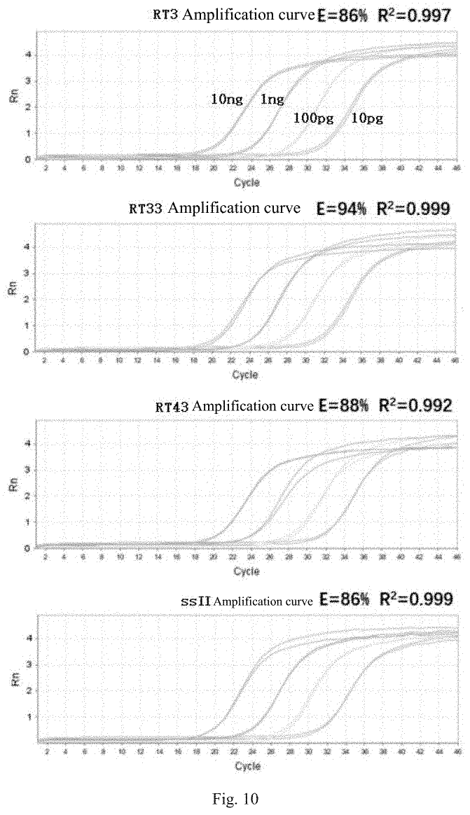

10 pg, 100 pg, 1 ng, and 10 ng of Hela total RNAs were each transcribed by using M-MLV RT reverse transcriptase mutants screened via polymerase activity, RNase H activity and thermal stability (RT3, RT6, RT33, RT40, RT41, RT43), along with the commercial ssII. The reaction system and conditions can refer to Table 9. Using the SYBR Green Ex Taq premix qPCR B2M gene for the reaction product cDNA, the curve with the logarithm of RNA input amount as the abscissa axis and the Ct value as the ordinate axis was drawn to calculate the efficiency of each reverse transcriptase and compare the sensitivity of reverse transcriptase (see FIG. 10).

The curves in each graph of FIG. 10 corresponds to the concentration of total RNA from left to right as 10 ng, 1 ng, 100 pg, and 10 pg. Each total RNA was measured in two parallel experiments, taking RT3 as an example, which has been marked in the graph. It can be seen from FIG. 10 that the sensitivity of RT33, RT43, RT3 as well as the commercial ssII is 10 pg total RNA.

### Example 8 Application Test and Analysis of M-MLV RT Reverse Transcriptase and its Mutants in Conventional RNA-Seq

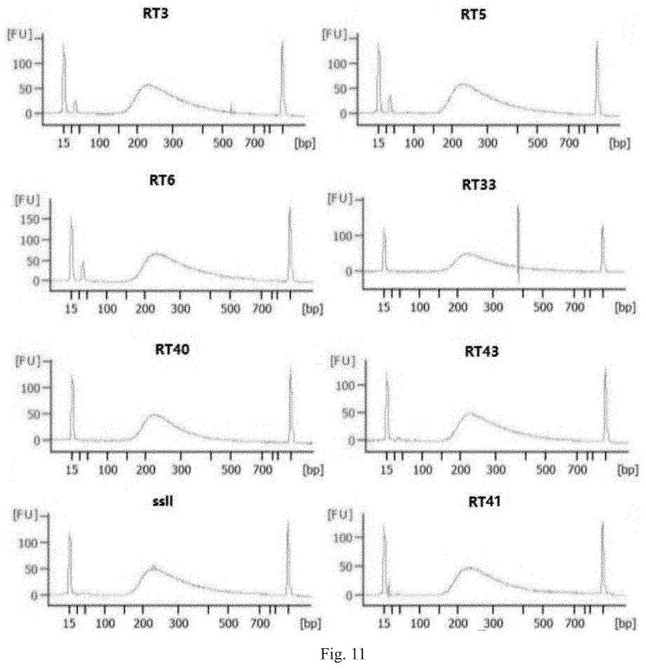

RNA-seq library construction was performed by using M-MLV RT reverse transcriptase mutants screened via polymerase activity, RNase H activity and thermal stability (RT3, RT5, RT6, RT33, RT40, RT41, RT43), along with the commercial ssII. Reverse transcriptase is used for reverse transcription of RNA. According to the instructions of MGI Easy mRNA Library Preparation Kit V2.0, the synthesized cDNA was subjected to end repair, adaptor addition, PCR enrichment, circularization and the like to construct a library, followed by machine sequencing. The yield and fragment distribution of cDNA PCR products were detected and compared by Aglient 2100 instrument to analyze the yield and fragment distribution of cDNA synthesized by reverse transcriptase (see FIG. 11 and Table 10). The transcription performance of reverse transcriptase mutants was compared through the machine sequencing results of library (see FIG. 9).

TABLE 10
Machine sequencing results of M-MLV RT mutants

| | | | | number of | | |
|---|---|---|---|---|---|---|
| | Filtering | | | gene or | | |
| | ratio | genome | gene set | transcript | | |
| | Project | comparison | comparison | detected | | |
| RNA-seq | Clean Reads | Total | Total | Total Gene | qPCR correlation | |
| RNA-seq | Ratio | Mapping Ratio | Mapping Ratio | Number | Spearman | Pearson |
| RT6 | 92.99% | 93.19% | 67.45% | 19635 | 0.862 | 0.851 |
| RT5 | 93.24% | 93.19% | 67.45% | 19635 | 0.862 | 0.851 |
| RT41 | 93.91% | 94.43% | 70.02% | 19694 | 0.868 | 0.859 |
| RT3 | 94.69% | 95.02% | 69.46% | 19674 | 0.863 | 0.853 |
| RT40 | 94.80% | 94.66% | 68.63% | 19685 | 0.861 | 0.855 |
| RT33 | 94.45% | 94.82% | 68.65% | 19666 | 0.862 | 0.855 |
| RT43 | 94.59% | 93.78% | 68.82% | 19696 | 0.866 | 0.857 |
| ssII | 94.25% | 94.34% | 68.50% | 19650 | 0.865 | 0.86 |

In Table 10, “Project clean reads ratio” represents available reads after filtering out reads containing adapters, low-quality reads and reads with too high N content. The first “Total Mapping ratio” represents genome comparison. The second “Total Mapping Ratio” represents the comparison of gene sets. “Total Gene number” represents the number of genes or transcripts detected. “Superman and Pearson” represent qPCR correlation.

FIG. 11 shows the cDNA yield and fragment distribution of different mutants in conventional RNA-seq. The results showed that RT3, RT5, RT6, RT33, RT40, R43 produced equivalent cDNA amount with the commercial enzyme in the conventional RNA-seq, and the fragments were distributed around 240 bp.

The results showed that libraries of RT40 and RT43 mutants exhibited better operating effects than the commercial enzyme ssII, and library of RT33 mutant had a similar operating effect to the commercial enzyme ssII.

### Example 9 Application Test and Analysis of M-MLV RT Reverse Transcriptase and its Mutants in Single-Cell RNA-Seq

MMLV RT has been widely used in cDNA library construction for single-cell sequencing, which uses the terminal transfer (TdT) activity of the enzyme, that is, a few of additional bases are added to the 3′ end of the blunt end of the newly generated cDNA complementary strand to be complementary with the 3′ end of template-switching oligonucleotide (TSO) added. However, this characteristic is negatively correlated with fidelity, and how to coordinate the relationship between the two characteristics to reach the best effect requires further research.

1. Detection of Function of Reverse Transcriptase Plus C Tail in Single-Cell RNA-Seq

The single-cell RNA-seq was conducted according to the method in the article (Full-length RNA-seq from single cells using Smart-seq2, Simone Picelli etal., Nature Protocols 9, 171-181(2014)). The reaction system and reaction conditions shown in Table 11 below are used to test the function of reverse transcriptase plus C tail in single-cell RNA-seq.

TABLE 11
C-tail plus reaction system and reaction conditions

| Component | Volume |
|---|---|
| RNA | 1 ul |
| OligodT30VN(100 uM) | 1 ul |
| 10 mM dNTP mix | 1 ul |
| reaction at 72° C. for 3 min | |
| Reverse transcriptase | 0.5 ul |
| RNase inhibitor (40U/μl) | 1 ul |
| first-strand buffer (5×) | 2 ul |
| DTT (100 mM) | 1 ul |
| Betaine (5M) | 2 ul |
| MgCl2 (50 mM) | 1.2 ul |
| TSO (100 uM) | 0.1 |
| 42° C., 90 min; | |
| KAPA HiFi HotStart ReadyMix (2×) | 12.5 ul |
| IS primer | 0.25 |

98° C., 3 min; 98° C., 20 s, 67° C., 15 s, 72° C.,
6 min (18 cycle); 72° C., 5 min

2. Library Construction Test of Single-Cell RNA-Seq

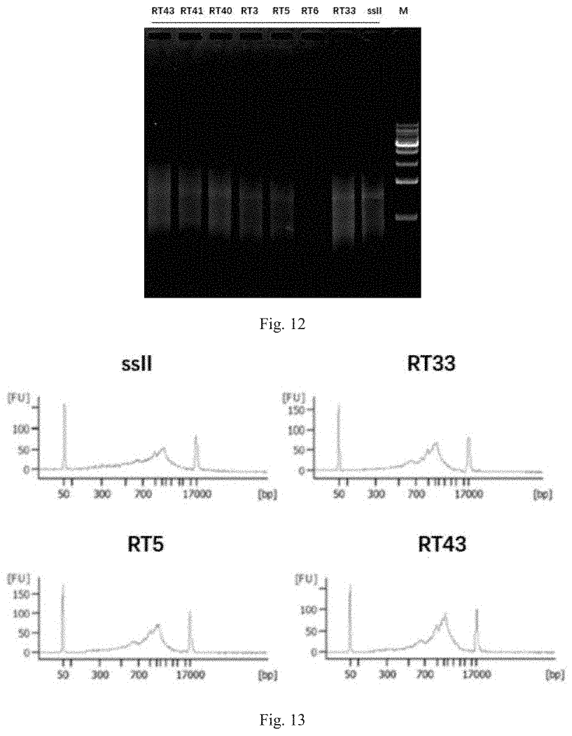

The above reaction system and principle were used to construct an RNA library, and the results of cDNA yield and fragment distribution of mutants are shown in FIG. 12.

The results showed that RT43, RT41, RT3, RT5, RT6, and RT33 all have the function of adding C tail. Among them, the function of adding C tail of RT6 is weaker than that of commercial enzyme ssII, and the function of adding C tail of other mutants is equivalent to that of ssII. The results in FIG. 13 show that cDNAs transcribed by RT33, RT5 and RT43 generally have a length of 2 k, and have the yield slightly higher than that of the commercial enzyme ssII in the single-cell RNA-seq.

In the description of this specification, descriptions with reference to the terms “one embodiment”, “some embodiments”, “examples”, “specific examples”, “some examples” or the like mean that a particular feature, structure, material, or characteristic described in connection with the embodiment or example is included in at least one embodiment or example of the present disclosure. In this specification, the schematic representations of the above-mentioned terms are not necessarily directed to the same embodiment or example. Moreover, the described particular feature, structure, material, or characteristic may be combined in any one or more embodiments or examples in a suitable manner. Furthermore, the different embodiments or examples and the features of the different embodiments or examples described in this specification may be combined by those skilled in the art without contradiction.

Although embodiments of the present disclosure have been shown and described in the above, it would be appreciated that the above embodiments are exemplary which cannot be construed to limit the present disclosure, and changes, alternatives, substitution and modifications can be made in the embodiments by those skilled in the art without departing from scope of the present disclosure.

What is claimed is:

1. A reverse transcriptase, comprising amino acid mutations at positions 302 and 524 compared to the amino acid sequence of SEQ ID NO: 2 of a wild-type M-MLV reverse transcriptase.

2. The reverse transcriptase according to claim 1, wherein the reverse transcriptase further comprises at least one of amino acid mutations at positions 286, 313, 330, 435, 562 and 583, compared to the amino acid sequence of SEQ ID NO: 2.

3. The reverse transcriptase according to claim 1, wherein the amino acid mutations comprises:
substitution of Glutamicacid at positions 302 with Lysine, and
substitution of Asparticacid at positions 524 with Alanine.

4. The reverse transcriptase according to claim 2, wherein the at least one of amino acid mutations at positions 286, 313, 330, 435, 562 and 583 comprises:
substitution of E at position 286 with K,
substitution of W at position 313 with F,
substitution of T at position 330 with P,
substitution of L at position 435 with R or G,
substitution of E at position 562 with Q, and
substitution of D at position 583 with G.

5. The reverse transcriptase according to claim 1, comprising at least one of mutations from E286K-E302K-W313F-D524A-D583G, E302K-D524A, E302K-L435R-D524A, E302K-L435R-D524A-E562Q, E302K-L435G-D524A, E286K-E302K-W313F-T330P-D524A-D583G, and E286K-E302K-D524A, compared to the amino acid sequence of SEQ ID NO: 2.

6. The reverse transcriptase according to claim 1, wherein the reverse transcriptase has increased polymerase activity, increased thermal stability and decreased RNase H activity.

7. The reverse transcriptase according to claim 1, wherein a polymerase activity of the reverse transcriptase is at least 1 to 4 times higher than that of the wild-type M-MLV reverse transcriptase.

8. The reverse transcriptase according to claim 1, wherein an RNase H activity of the reverse transcriptase is reduced by 30% to 80% compared to that of the wild-type M-MLV reverse transcriptase.

9. The reverse transcriptase according to claim 1, wherein the reverse transcriptase keeps its reverse transcriptase activity unchanged after being heated at 50° C. for 30 minutes, or wherein the reverse transcriptase keeps its reverse transcriptase activity unchanged after being heated at 42° C. for 30 minutes.

10. An isolated nucleic acid molecule encoding the reverse transcriptase of claim 1.

11. A construct comprising the isolated nucleic acid molecule of claim 10,
preferably the isolated nucleic acid molecule is operably linked to a promoter,
wherein the promoter is one selected from λ-PL promoter, tac promoter, trp promoter, araBAD promoter, T7 promoter and trc promoter.

12. A host cell comprising the construct of claim 11.

13. A method for producing a reverse transcriptase of claim 1, comprising:
culturing a host cell, wherein the host cell comprises a construct comprising the isolated nucleic acid molecule encoding the reverse transcriptase,
preferably the isolated nucleic acid molecule is operably linked to a promoter,
wherein the promoter is one selected from λ-PL promoter, tac promoter, trp promoter, araBAD promoter, T7 promoter and trc promoter,
inducing the host cell to express the reverse transcriptase, and
isolating the reverse transcriptase,
preferably the host cell is Escherichia coli.

14. A kit comprising the reverse transcriptase of claim 1,
preferably the kit further comprises at least one from one or more nucleotides, one or more DNA polymerases, one or more buffers, one or more primers, and one or more terminators,
wherein the terminator is dideoxynucleotide.

15. A method for reverse transcription of nucleic acid molecules, comprising:
mixing at least one nucleic acid template with at least one reverse transcriptase to obtain a mixture, wherein the reverse transcriptase is the reverse transcriptase of claim 1,
subjecting the mixture to a reverse transcription reaction to obtain a first nucleic acid molecule, wherein the first nucleic acid molecule is completely or partially complementary to the at least one nucleic acid template,
wherein the first nucleic acid molecule is a cDNA molecule,
wherein the nucleic acid template is mRNA,
preferably wherein an amount of the nucleic acid template is at least 10 pg.

16. The method according to claim 15, further comprising:
subjecting the first nucleic acid molecule to a PCR reaction, to obtain a second nucleic acid molecule,
wherein the second nucleic acid molecule is completely or partially complementary to the first nucleic acid molecule.

17. A method for amplifying nucleic acid molecules, comprising:
subjecting at least one nucleic acid template and at least one reverse transcriptase to a first mixing reaction, to obtain a reaction product, wherein the at least one reverse transcriptase is the reverse transcriptase of claim 1,
subjecting the reaction product and at least one DNA polymerase to a second mixing reaction, to obtain an amplified nucleic acid molecule, wherein the amplified nucleic acid molecule is completely or partially complementary to the at least one nucleic acid template.

18. The method according to claim 17, further comprising:
sequencing the amplified nucleic acid molecule to determine a nucleotide sequence of the amplified nucleic acid molecule.

19. A method for constructing a cDNA library, comprising:
subjecting a biological sample to be tested to RNA extraction, to obtain mRNA of the biological sample to be tested,
treating the mRNA of the biological sample to be tested by the method of claim 15, to obtain cDNA molecules, and
subjecting the cDNA molecules to amplification and library construction to obtain a cDNA library.

20. The method according to claim 19, wherein the biological sample to be tested is an animal tissue, a plant tissue or bacteria,
preferably wherein a total RNA content in the biological sample to be tested is at least 10 pg,
preferably wherein the biological sample to be tested is at least one selected from soil, feces, blood and serum,
preferably wherein a length of obtained cDNA is at least 2000 bp.


SEQUENCE LISTING (4 sequences)

SEQ ID NO: 1 - DNA, 2016 bp, Artificial Sequence
  wild-type M-MLV RT
atgctgaacatcgaggacgaacaccgtctgcacgagaccagcaaggaaccggacgtgagcctgggtagcacctggctgagcgatttcccgcaggcgtgggcggagaccggtggcatgggtctggcggtgcgtcaagcgccgctgatcattccgctgaaggcgaccagcaccccggttagcatcaaacagtacccgatgagccaagaagcgcgtctgggtatcaaaccgcacattcagcgtctgctggaccaaggcattctggttccgtgccaaagcccgtggaacaccccgctgctgccggtgaagaaaccgggcaccaacgactatcgtccggttcaggatctgcgtgaggtgaacaagcgtgttgaagatatccacccgaccgtgccgaacccgtacaacctgctgagcggtctgccgccgagccatcagtggtataccgttctggacctgaaagatgcgttcttttgcctgcgtctgcatccgaccagccagccgctgtttgcgtttgagtggcgtgacccggaaatgggtattagcggtcagctgacctggacccgtctgccgcaaggcttcaagaacagcccgaccctgtttgacgaggcgctgcaccgtgacctggcggattttcgtatccagcacccggatctgattctgctgcaatacgtggacgatctgctgctggcggcgaccagcgaactggattgccagcaaggtacccgtgcgctgctgcagaccctgggtaacctgggctatcgtgcgagcgcgaagaaagcgcaaatctgccagaagcaagtgaaatacctgggttatctgctgaaagagggtcagcgttggctgaccgaggcgcgtaaggaaaccgttatgggtcagccgaccccgaaaaccccgcgtcaactgcgtgagttcctgggtaccgcgggcttttgccgtctgtggattccgggttttgcggaaatggcggcgccgctgtacccgctgaccaaaaccggtaccctgtttaactggggcccggaccagcaaaaggcgtatcaggaaattaaacaagcgctgctgaccgcgccggcgctgggtctgccggacctgaccaagccgttcgagctgtttgtggatgaaaagcagggttacgcgaaaggcgttctgacccaaaaactgggtccgtggcgtcgtccggtggcgtatctgagcaagaaactggacccggttgcggcgggttggccgccatgcctgcgtatggtggcggcgatcgcggttctgaccaaggatgcgggtaaactgaccatgggtcagccgctggtgattctggcgccgcacgcggtggaggcgctggttaaacaaccgccggatcgttggctgagcaacgcgcgtatgacccactaccaggcgctgctgctggacaccgatcgtgttcaatttggtccggtggttgcgctgaacccggcgaccctgctgccgctgccggaggaaggtctgcagcacaactgcctggacattctggcggaggcgcatggtacccgtccggacctgaccgatcaaccgctgccggacgcggatcacacctggtataccgatggtagcagcctgctgcaggaaggtcagcgtaaagcgggtgcggcggtgaccaccgagaccgaagttatctgggcgaaggcgctgccggcgggtaccagcgcgcagcgtgcggagctgattgcgctgacccaagcgctgaagatggcggaaggcaagaaactgaacgtttacaccgacagccgttatgcgttcgcgaccgcgcacatccacggcgagatttaccgtcgtcgtggtctgctgaccagcgagggcaaggaaatcaagaacaaggatgaaatcctggcgctgctgaaggcgctgtttctgccgaaacgtctgagcatcattcactgcccgggtcaccagaaaggtcacagcgcggaggcgcgtggtaaccgtatggcggaccaagcggcgcgtaaagcggcgatcaccgaaaccccggataccagcaccctgctgatt

SEQ ID NO: 2 - protein, 672 aa, Artificial Sequence
  wild-type M-MLV RT
MLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 3 - DNA, 30 bp, Artificial Sequence
  single-stranded DNA Poly (dT) 30
  misc_feature (1)..(1): 5′ fluorescent group cy3
tttttttttttttttttttttttttttttt

SEQ ID NO: 4 - DNA, 30 bp, Artificial Sequence
  single-stranded RNA Poly(rA) 30
  misc_feature (30)..(30): BHQ2 quenching group
aaaaaaaaaaaaaaaaaaaaaaaaaaaaaa